(19) United States
(12) Patent Application Publication
(10) Pub. No.: US 2007/0249951 A1
(43) Pub. Date: Oct. 25, 2007

(54) Apparatus, System and Method for Assessing Alveolar Inflation

(75) Inventors: Gary Nieman, Manlius, NY (US); David Carney, Savannah, GA (US); Joseph DiRocco, Syracuse, NY (US)

Correspondence Address: BOND, SCHOENECK & KING, PLLC, ONE LINCOLN CENTER, SYRACUSE, NY, 13202-1355, US

(73) Assignee: The Research Foundation of State University of New York, Albany, NY (US)

(21) Appl. No.: 11/737,473

(22) Filed: Apr. 19, 2007

Related U.S. Application Data
(60) Provisional application No. 60/745,145, filed on Apr. 19, 2006.

(51) Int. Cl. A61B 5/08; A61M 16/00

(52) U.S. Cl. 600/532; 128/200.24; 128/204.23

(57) ABSTRACT

A system and method for measuring alveolar performance during ventilation using a microphone positioned adjacent to the distal end of a ventilation tube. The microphone is connected to a monitor that filters the signals received from the microphone to eliminate unwanted noise, such as that caused by the heart and turbulent airflow through the large airways of the lungs. The desired signals are then amplified and displayed on a graph as a function of lung volume during inflation. A clinician may take appropriate ventilation strategies based on the results displayed by the system to avoid ventilator induced lung injuries and decrease the mortality rates of patients having acute respiratory distress syndrome.

### PRIORITY CLAIM

The present application claims priority to U.S. Provisional Patent Application Ser. No. 60/745,145, filed Apr. 19, 2006, the entirety of which is hereby incorporated by reference.

### BACKGROUND OF THE INVENTION

1. Field of the Invention

The present invention relates to assessment of lung functions and, more specifically, to an apparatus, system and method for assessing alveolar inflation during ventilation using sound.

2. Description of the Prior Art

During mechanical ventilation, ventilator induced lung injuries (VILIs) can significantly increase the mortality rate of patients having acute respiratory distress syndrome (ARDS). VILIs may be reduced according to the convention methods by using low tidal volume ventilation or by setting positive end-expiratory pressure above the lower inflection point on the inflation limb of the whole lung pressure/volume curve. These methods are not infallible, however, as substantial alveolar recruitment above the lower inflection point may occur. In addition, higher levels of positive end-expiratory pressure may actually reduce VILIs.

The only know systems or method for visualizing the operation of alveoli require a Computer Axial Tomography (CAT) scan. These systems may not be employed at the bedside, and, as a result, fail to provide information in real-time that may be used by a clinician to adjust ventilation as a patient is being ventilated.

### SUMMARY OF THE INVENTION

It is therefore a principal object and advantage of the present invention to provide a method and system for reducing ventilator induced lung injuries.

It is a further object and advantage of the present invention to provide a method and system for determining alveolar opening and closing at the patient bedside.

It is an additional object and advantage of the present invention to provide a method and system for dynamically assessing alveolar inflation.

It is also an object and advantage of the present invention to provide a method and system for reducing patient mortality.

Other objects and advantages of the present invention will in part be obvious, and in part appear hereinafter.

In accordance with the foregoing objects and advantages, the system of the present invention comprises a microphone positioned at the tip of an endotracheal tube of a ventilator.

The microphone is connected to a noise analysis module for eliminating unwanted noise, such as that caused by the heart and turbulent airflow through the large airways of the lungs.

The noise analysis module filters out all signals except the frequency of the sounds (S) created by alveolar opening and closing during ventilation. The filtered alveolar opening sounds are then amplified and displayed on a graph along with lung volume (V) during inflation. Normal lung alveoli will make very little sound during inflation and generate a relatively flat V/S graph, while the alveoli of a patient having acute respiratory distress syndrome will be collapsed and “sticky,” therefore producing a distinct noise that results in a jagged V/S graph. Thus, if a clinician perceives a jagged line, protective ventilation strategies may be taken to return the V/S graph to normal, i.e., to produce a smooth line representative of normal alveolar action. A smooth line will indicate that all alveoli are open and stable such that ventilator induced lung injury (VILI) will be minimized which will greatly reducing the morbidity and mortality associated with ARDS.

### BRIEF DESCRIPTION OF THE DRAWINGS

The present invention will be more fully understood and appreciated by reading the following Detailed Description in conjunction with the accompanying drawings, in which:

FIG. 1 is schematic of a system according to the present invention.

FIG. 2 is a schematic of a noise analysis module according to the present invention.

FIG. 3 is a graph of alveolar noise according to the present invention.

### DETAILED DESCRIPTION

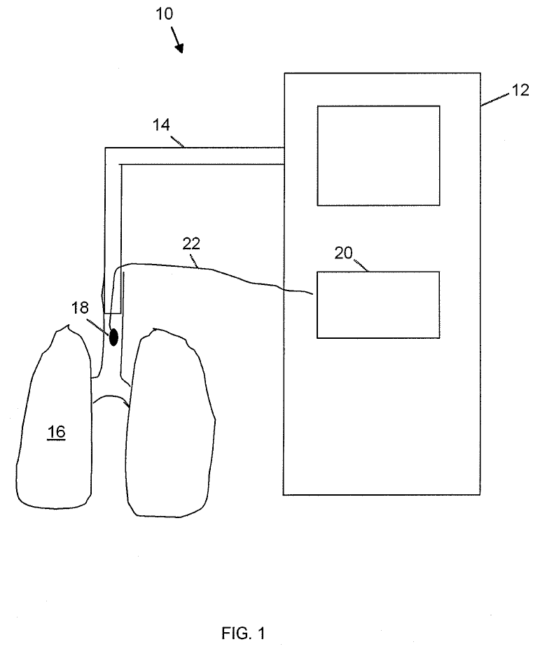

Referring now to the drawings, wherein like reference numerals refer to like parts throughout, there is seen in FIG. 1 an alveolar noise detection system 10 according to the present invention. System 10 is preferably used in connection with a conventional ventilator 12 having a ventilator tube 14 extending therefrom that is adapted for positioning in the lungs 16 of a patient. System 10 comprises a microphone 18 positioned adjacent to the distal tip 20 of tube 14. Microphone 18 is interconnected to a monitor 20 via conventional means, such as a wire 22. It should be understood that microphone 18 may instead transmit data wirelessly to monitor 20 using any number of conventional wireless protocols.

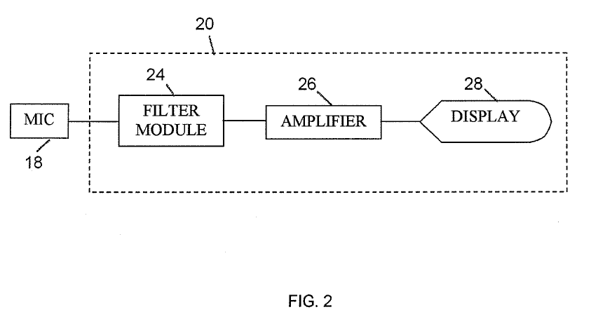

Referring to FIG. 2, monitor 20 comprises a filter module 24 for receiving signals from microphone 18 and eliminating unwanted noise from the signals, such as the frequencies caused by the heart and the turbulent airflow through the large airways of the lungs. Filter module 24 is tuned to filter out all signals except the frequency or frequencies of the sounds of the opening and closing of the alveoli in lungs 16. See, e.g., Z. Hantos et al., Acoustic Evidence of Airway Opening During Recruitment in Excised Dog Lungs, Journal of Applied Physiology, V. 97, pp. 592-598 (2004), hereby incorporated by reference.

Monitor 20 further comprises an amplifier 26 interconnected to filter module 24 for increasing the strength of the signals output from filter module 24. Monitor 20 also comprises a display 28 interconnected to amplifier 26 for plotting the remaining signals as a graph visible to a clinician or health services provider. It should be obvious to one of skill in the art that the various modules of monitor 20 may be implemented digitally, such as in a programmable microcontroller, or through conventional analog circuitry.

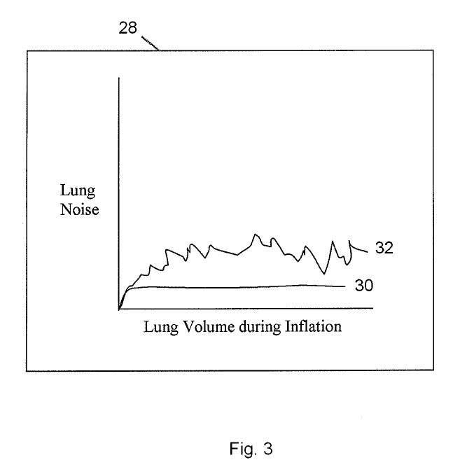

Referring to FIG. 3, the signals from monitor 20 are preferably plotted as the level of noise from the alveoli verses lung volume during inflation. As normal lung alveoli will make very little noise during inflation, the resulting graph 30 will be relatively flat. The alveoli of a patient having acute respiratory distress syndrome will be collapsed and “sticky,” therefore producing a jagged graph 32. A clinician may therefore take appropriate ventilation strategies to return jagged graph 32 into flat graph 30 while monitoring the breath-by-breath, real-time output of alveolar sounds by system 10.

What is claimed is:

1. A system for assessing alveolar inflation in a patient during ventilation, comprising:
a. a tube having proximal and distal ends and adapted for positioning within a patient's trachea;
b. a microphone interconnected to said ventilation tube and adapted for positioning adjacent said distal end of said tube, said microphone further adapted to capture sounds and output a signal representative of said sounds;
c. a filter interconnected to said microphone and adapted to receive and filter the signals output from said microphone, said filter further adapted to output a signal within a predetermined frequency range that is representative of certain lung noise; and
d. a display for displaying data represented by said filtered signal.

2. The system according to claim 1 wherein said predetermined frequency range captures sounds created by alveolar opening and closing during ventilation of the patient.

3. The system according to claim 1, further comprising an amplifier interconnected to said filter and adapted to receive and amplify said signal within the predetermined frequency range.

4. The system according to claim 1, further comprising said display being adapted to graphically display said filtered signal relative to lung volume.

5. The system according to claim 1, wherein said tube is an endotracheal tube.

6. The system according to claim 1, wherein said microphone is electrically connected to said filter.

7. The system according to claim 1, wherein said microphone is adapted to wirelessly transmit said signal with the predetermined frequency range to said filter which is adapted to wirelessly receive said signal with the predetermined frequency range.

8. A method for assessing alveolar inflation in a patient during ventilation by listening to sounds produced by the patient's lungs, comprising the steps of:
a. providing a tube adapted to be placed within a patient's trachea and a microphone that is positioned adjacent the distal end of said tube;
b. actuating said microphone while ventilating the patient, wherein said microphone is adapted to output a signal representative of the sounds;
c. providing a filter that is adapted to receive said signal representative of the sounds and filter said signal to output a filtered signal representing sounds in a predetermined frequency range;
d. displaying data represented by said filtered signal.

9. The method according to claim 8, comprising the further step of amplifying said filtered signal prior to displaying data.

10. The method according to claim 8, wherein said step of providing a filter comprises a filter adapted to capture sounds created by alveolar opening and closing during ventilation of the patient.

11. An endotracheal tube used for ventilating a patient, comprising:
a. a tube having proximal and distal ends; and
b. a microphone interconnected to said tube and adapted for positioning adjacent said distal end.
